(19) United States
(12) Patent Application Publication
(10) Pub. No.: US 2026/0090754 A1
(43) Pub. Date: Apr. 2, 2026

(54) MEDICAL IMAGE PROCESSING APPARATUS

(71) Applicant: CANON MEDICAL SYSTEMS CORPORATION, Otawara-shi (JP)

(72) Inventors: Ryo SHIROISHI, Kawasaki (JP); Takuya SAKAGUCHI, Utsunomiya (JP)

(73) Assignee: CANON MEDICAL SYSTEMS CORPORATION, Otawara-shi (JP)

(21) Appl. No.: 19/324,359

(22) Filed: Sep. 10, 2025

(30) Foreign Application Priority Data
Sep. 30, 2024 (JP) 2024-170751

(51) Int. Cl. A61B 5/294; A61B 5/055; A61B 34/10; G01R 33/48

(52) U.S. Cl. CPC A61B 5/294; A61B 5/055; A61B 34/10; G01R 33/4806; A61B 2034/105; A61B 2034/107

(57) ABSTRACT

A medical image processing apparatus according to an embodiment includes a vessel diameter image generation unit, a distance image generation unit, a candidate placement position identification unit, and a display control unit. The vessel diameter image generation unit generates a vessel diameter image indicating a size of a vessel diameter in a brain, from a brain blood vessel image obtained by capturing an image of a blood vessel of the brain. The distance image generation unit identifies a target position corresponding to a nerve activity, which is a measurement target in the brain, based on an image related to at least either one of a function region of the brain and arrangement of a nerve in the brain, and generates a distance image indicating a distance from the identified target position. The candidate placement position identification unit identifies a position with both of a vessel diameter and a distance from the target position satisfying a prescribed standard on the blood vessel, based on the vessel diameter image and the distance image, as a candidate placement position of a device. The display control unit displays the identified candidate placement position in an intensified manner on an image indicating the brain.

### CROSS-REFERENCE TO RELATED APPLICATIONS

This application is based upon and claims the benefit of priority from Japanese Patent Application No. 2024-170751, filed September 30, 2024, the entire contents of which are incorporated herein by reference.

### FIELD

Embodiments described herein relate generally to a medical image processing apparatus.

### BACKGROUND

As medical treatment for compensating for a motor function of a patient with a disability in a motor function due to a problem in neurotransmission as in amyotrophic lateral sclerosis (ALS), there is a brain computer interface (BCI) that controls an artificial arm or an artificial leg by directly collecting nerve activity data of a brain.

For example, as a method of collecting nerve activity data of a brain, there is a method of placing a sheet-like electrode (also called a cortical electrode) on the surface of the brain. Furthermore, as a new method, a technique of placing a stent-like electrode (also called intravascular electrode) in a blood vessel via a catheter to collect nearby neural activity data has been studied. By the technique of the intravascular electrode, it is possible to install an electrode in a cranium by a safe method of placing a device in a blood vessel, which is an established method in the cardiovascular field. This is advantageous in that an electrode can be placed in a cranium with lower invasiveness than that in a conventional method of placing a cortical electrode on the brain surface by a craniotomy procedure. The sensitivity of the intravascular electrode is lower as compared with that of the cortical electrode because a potential difference generated by a nerve activity of a brain and transmitted through surrounding brain tissue and vascular tissue is measured. To suppress a decline in sensitivity, an intravascular electrode is to be placed as close as possible to an occurrence location of a measurement target nerve activity. In addition, because it is dangerous to insert a catheter into a fine blood vessel, it is necessary to place an intravascular electrode by selecting a sufficiently-wide blood vessel.

Nevertheless, because a vascular network surrounding the brain has a complicated shape, and varies in inner diameter, it has been not easy for a user to determine a placement position of an intravascular electrode in consideration of both of which blood vessel is closer to a measurement target and which blood vessel is catheter-insertable.

### BRIEF DESCRIPTION OF THE DRAWINGS

FIG. 1 is a diagram illustrating the overview of input-output of processing of a medical image processing apparatus according to a first embodiment; FIG. 2 is a diagram illustrating an example of a configuration of the medical image processing apparatus according to the first embodiment; FIG. 3 is a diagram illustrating an example of a brain blood vessel image and a vessel diameter image according to the first embodiment; FIG. 4 is a diagram illustrating an example of a brain function image and a brain function distance image according to the first embodiment; FIG. 5 is a diagram illustrating an example of a brain function image including two activation regions, and a brain function distance image according to the first embodiment; FIG. 6 is a diagram illustrating an example of a nerve fiber image and a nerve fiber end point distance image according to the first embodiment; FIG. 7 is a diagram illustrating an example of a candidate placement position display image in which a brain blood vessel image according to the first embodiment is used; FIG. 8 is a diagram illustrating an example of a candidate placement position display image in which a vessel diameter image according to the first embodiment is used; FIG. 9 is a diagram illustrating an example of a candidate placement position display image in which a brain function image according to the first embodiment is used; FIG. 10 is a diagram illustrating an example of a candidate placement position display image in which a nerve fiber image according to the first embodiment is used; FIG. 11 is a flowchart illustrating an example of a flow of identification processing of a candidate placement position of an intravascular electrode according to the first embodiment; FIG. 12 is a diagram illustrating the overview of input-output of processing of a medical image processing apparatus according to a second embodiment; FIG. 13 is a diagram illustrating an example of a configuration of the medical image processing apparatus according to the second embodiment; FIG. 14 is a flowchart illustrating an example of a flow of identification processing of a candidate placement position of an intravascular electrode according to the second embodiment; FIG. 15 is a diagram illustrating the overview of input-output of processing of a medical image processing apparatus according to a third embodiment; FIG. 16 is a diagram illustrating an example of a configuration of the medical image processing apparatus according to the third embodiment; FIG. 17 is a flowchart illustrating an example of a flow of identification processing of a candidate placement position of an intravascular electrode according to the third embodiment; FIG. 18 is a diagram illustrating the overview of input-output of processing of a medical image processing apparatus according to a fourth embodiment; FIG. 19 is a diagram illustrating an example of a configuration of the medical image processing apparatus according to the fourth embodiment; FIG. 20 is a diagram illustrating an example of a brain region atlas and a brain region distance image according to the fourth embodiment; and FIG. 21 is a flowchart illustrating an example of a flow of identification processing of a candidate placement position of an intravascular electrode according to the fourth embodiment.

### DETAILED DESCRIPTION

A medical image processing apparatus according to an embodiment includes a vessel diameter image generation unit, a distance image generation unit, a candidate placement position identification unit, and a display control unit. The vessel diameter image generation unit generates a vessel diameter image indicating a size of a vessel diameter in a brain, from a brain blood vessel image obtained by capturing an image of a blood vessel of the brain. The distance image generation unit identifies a target position corresponding to a nerve activity being a measurement target in the brain, based on an image related to at least either one of a function region of the brain and arrangement of a nerve in the brain, and generates a distance image indicating a distance from the identified target position. The candidate placement position identification unit identifies a position with both of a vessel diameter and a distance from the target position satisfying a prescribed standard on the blood vessel, based on the vessel diameter image and the distance image, as a candidate placement position of a device. The display control unit displays the identified candidate placement position in an intensified manner on an image indicating a brain.

Various Embodiments will be described hereinafter with reference to the accompanying drawings.

### First Embodiment

Hereinafter, embodiments of a medical image processing apparatus will be described in detail with reference to the drawings. A medical image processing apparatus according to the present embodiment is used when a placement position of an intravascular electrode is determined. The intravascular electrode is a stent-like electrode, and is percutaneously inserted by a catheter and placed in a blood vessel of a brain of a subject (patient) to collect nerve activity data of the brain. The intravascular electrode measures a potential difference generated by a nerve activity of the brain and transmitted through surrounding brain tissue and vascular tissue. It accordingly becomes possible to collect nerve activity data near a position where the intravascular electrode is placed. The intravascular electrode serves as an example of a device in the present embodiment.

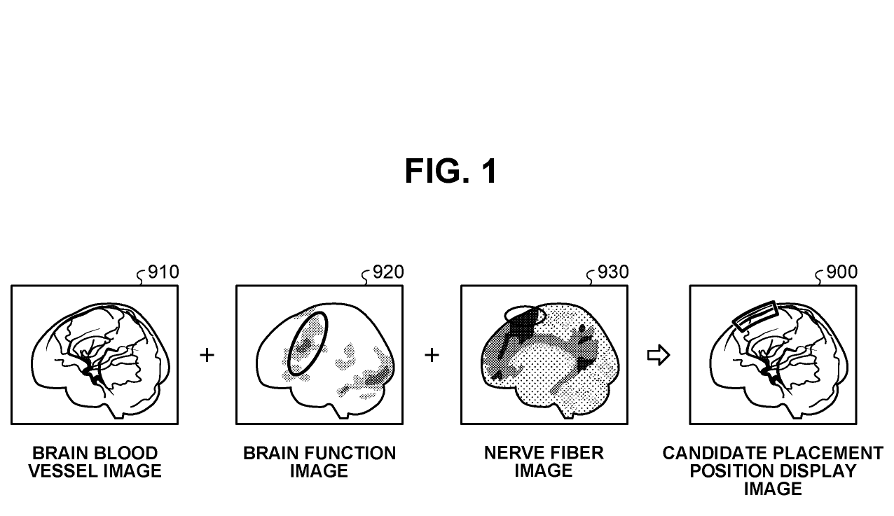

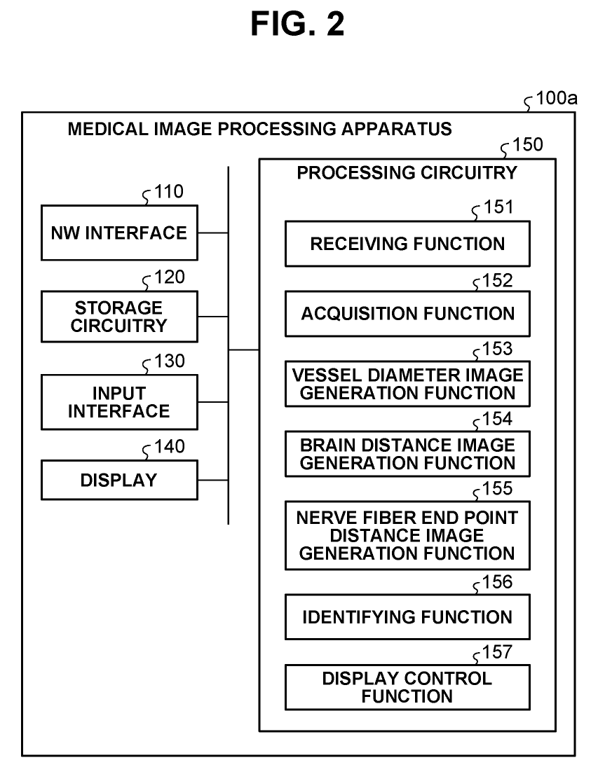

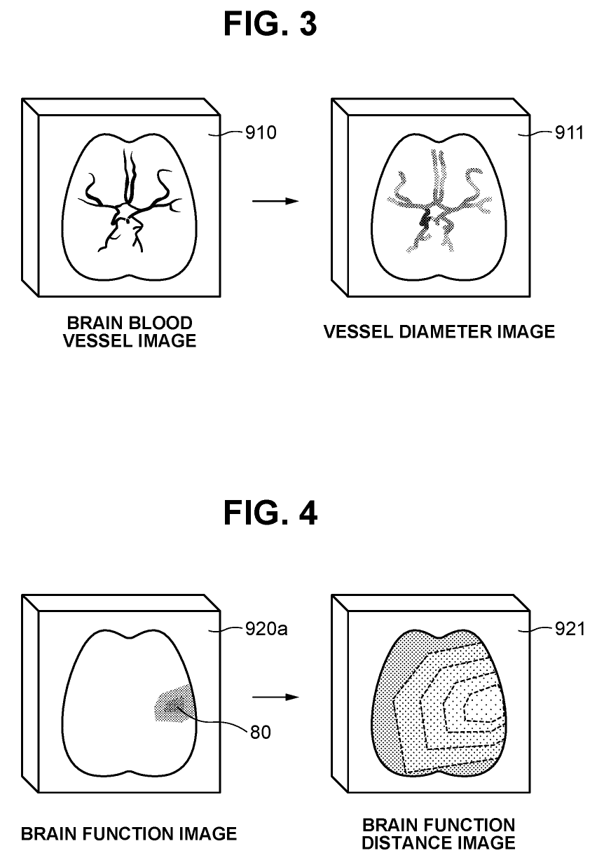

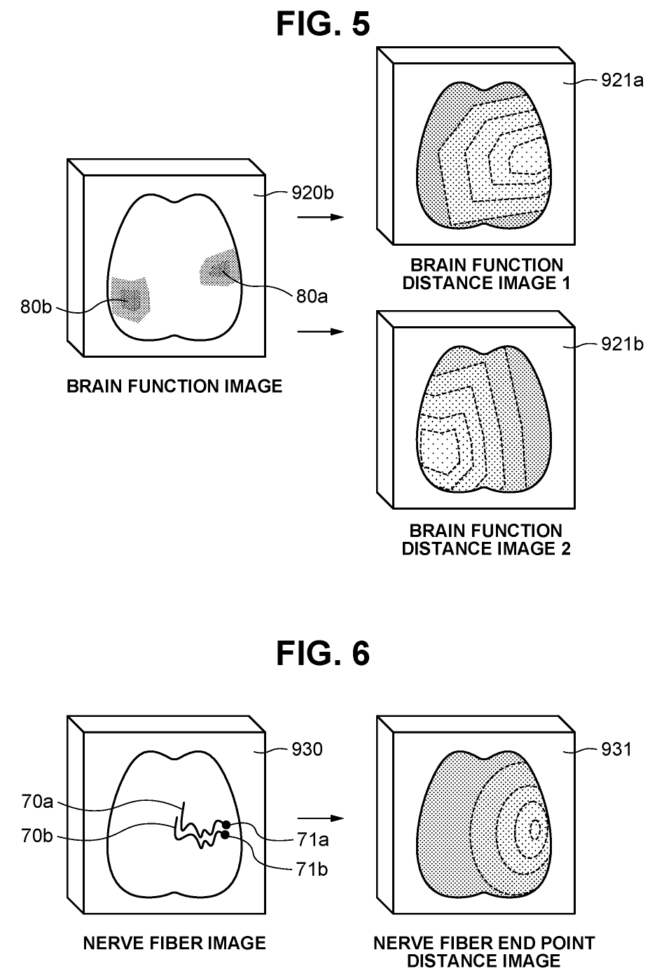

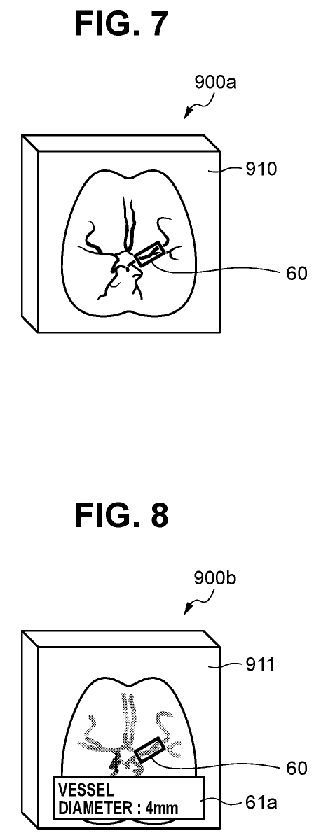

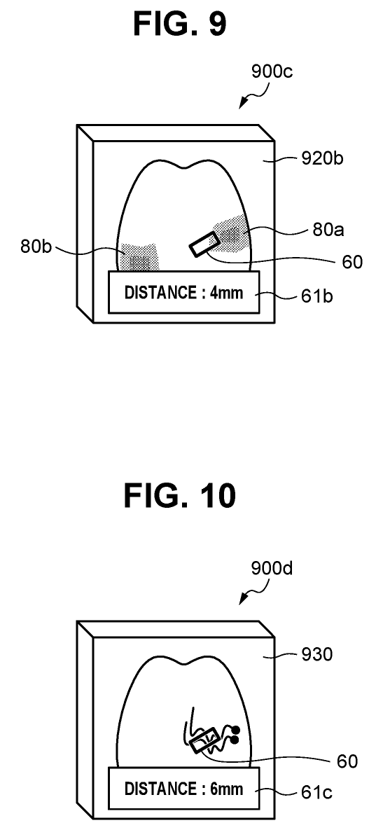

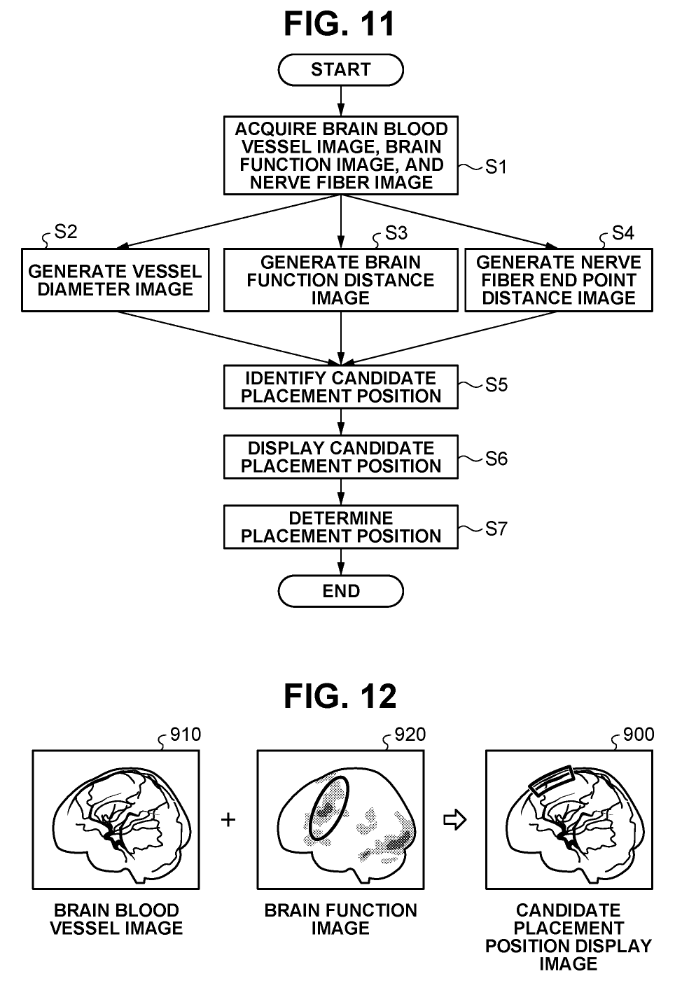

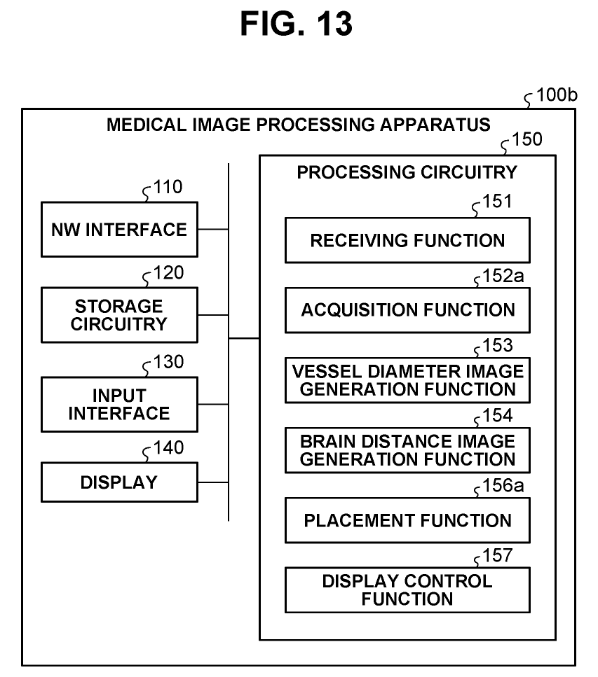

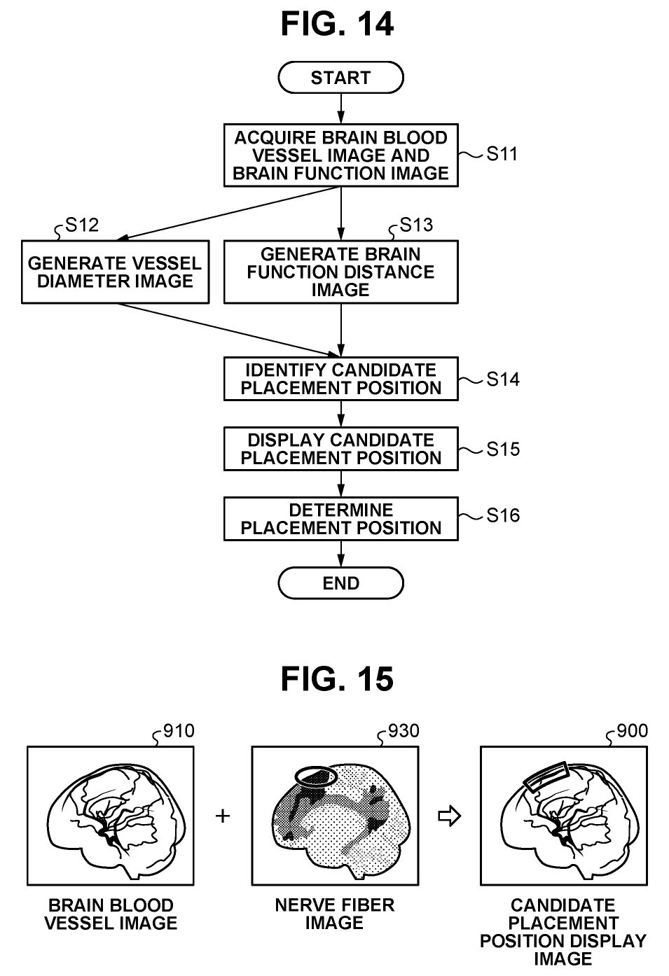

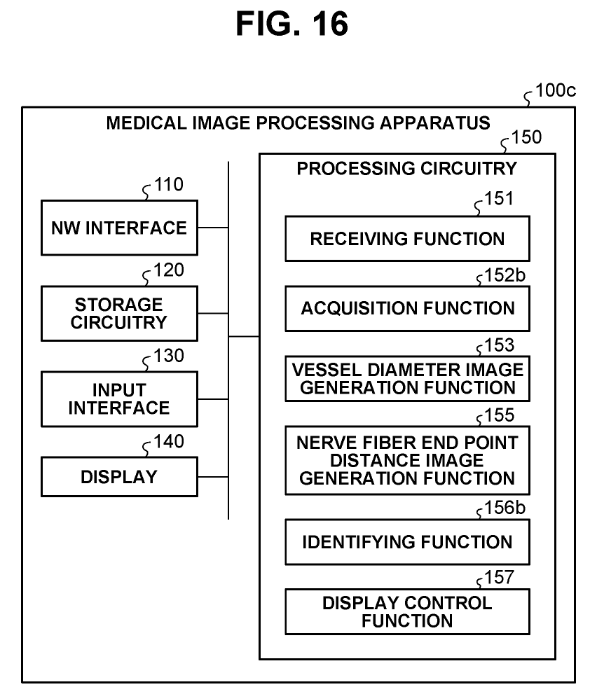

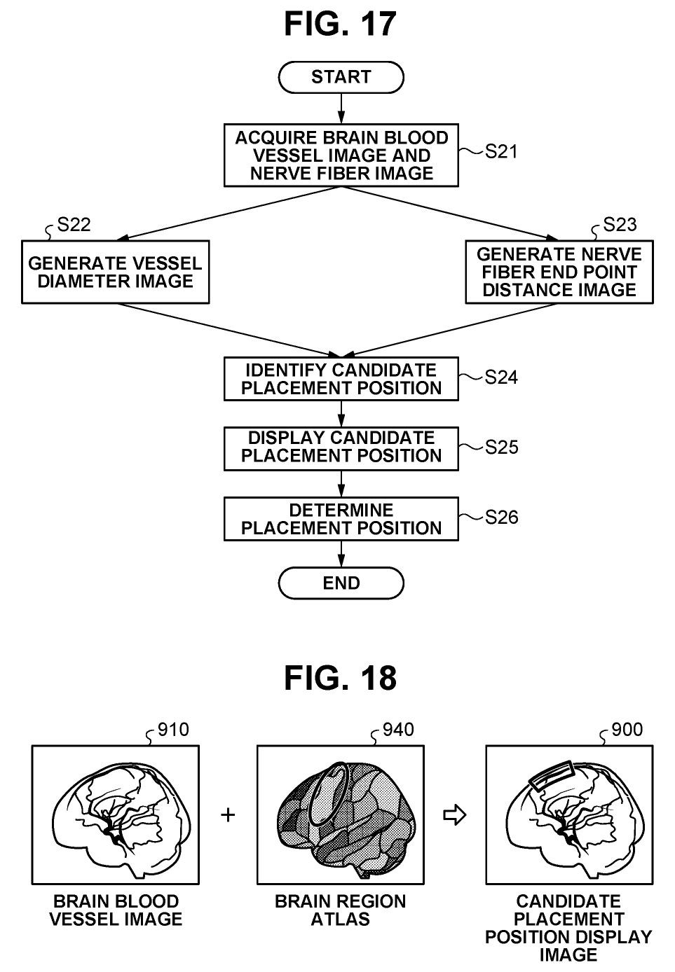

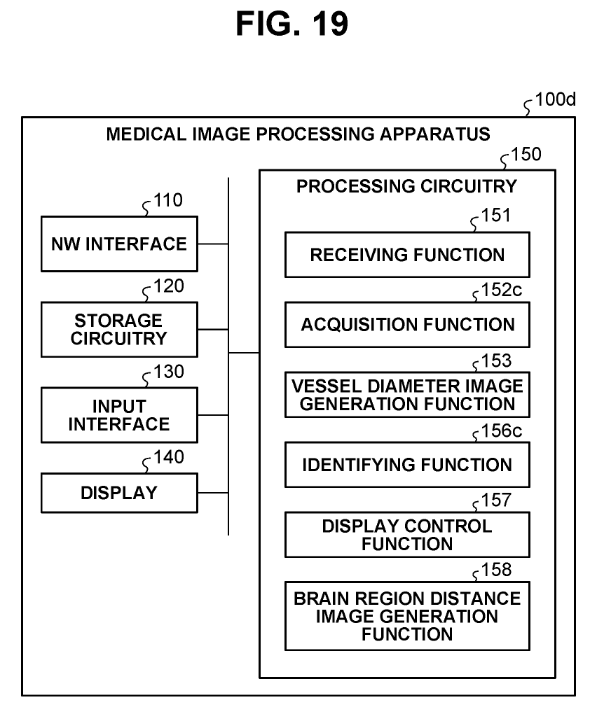

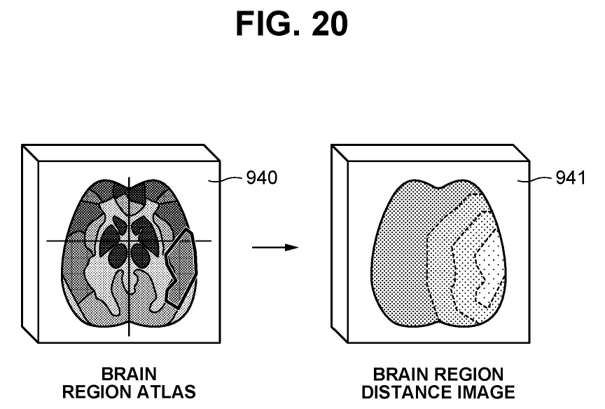

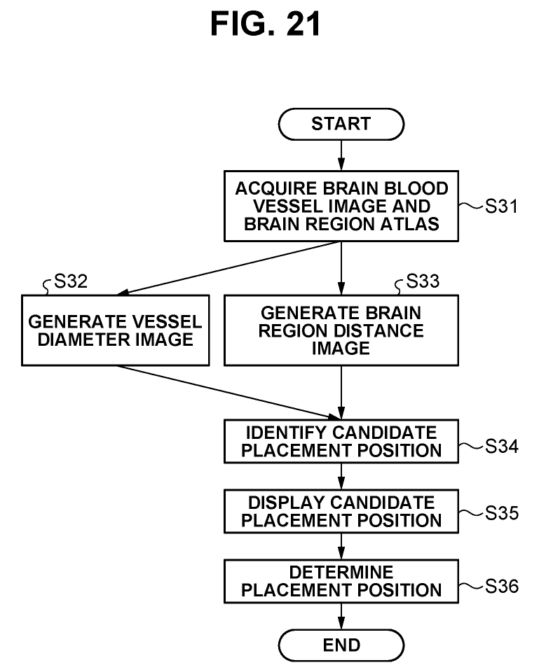

FIG. 1 is a diagram illustrating the overview of input-output of processing of a medical image processing apparatus according to the first embodiment. As illustrated in FIG. 1, the medical image processing apparatus according to the present embodiment outputs a candidate placement position display image 900 using a brain blood vessel image 910, a brain function image 920, and a nerve fiber image 930 as inputs.

The brain blood vessel image 910 is an image in which a blood vessel of a brain of a subject from which a nerve activity is to be measured is visualized. In the blood vessel of the brain of the subject, an intravascular electrode for measuring the nerve activity is to be placed. Various medical images can be employed as the brain blood vessel image 910. For example, in a case where a doctor indwells an intravascular electrode into an artery, an image captured by computed tomography (CT) angiography, magnetic resonance imaging (MRI) angiography, digital subtraction angiography (DSA), or the like is used as the brain blood vessel image 910. In addition, in a case where a doctor indwells an intravascular electrode into a vein, an image captured by MR venography or the DSA is used as the brain blood vessel image 910.

The brain function image 920 is an image in which an activation region of a nerve activity desired to be measured by the intravascular electrode is visualized. For example, in a case where a nerve activity to be measured is an activity that occurs when a hand finger or an ankle of a subject operates, the brain function image 920 is a functional MRI (fMRI) image or the like that is captured when a subject performs a curvature movement task of a hand finger or an ankle. Alternatively, in a case where a nerve activity to be measured is an activity that occurs when a subject imagines himself/herself consciously moving a hand finger or an ankle, the brain function image 920 is an fMRI image or the like that is captured when a subject imagines himself/herself consciously moving a hand finger or an ankle. The brain function image 920 is captured while a subject is performing an operation or imagining related to a nerve activity to be measured.

The nerve fiber image 930 is an image in which a nerve fiber in the brain is visualized, and specifically, is a tractography of the MRI.

The candidate placement position display image 900 is an image in which a candidate placement position of an intravascular electrode is displayed in an intensified manner on an image indicating the brain. A doctor or the like checks the candidate placement position display image 900 and determines a placement position of the intravascular electrode. A candidate placement position of the intravascular electrode is a position that exists in a blood vessel with a vessel diameter sufficiently large to place the intravascular electrode, in a blood vessel region of the brain, and is close to an activation region of a brain function to be measured and an end point of a nerve fiber. A method of identifying a position where the intravascular electrode can be placed will be described below.

In the present embodiment, the brain blood vessel image 910, the brain function image 920, the nerve fiber image 930, and the candidate placement position display image 900 are assumed to be three-dimensional image data (volume data), for example. Part or all of these may be two-dimensional image data.

FIG. 2 is a diagram illustrating an example of a configuration of a medical image processing apparatus 100a according to the first embodiment. The medical image processing apparatus 100a is a server or a computer such as a personal computer (PC), for example.

As illustrated in FIG. 2, the medical image processing apparatus 100a includes, for example, a network (NW) interface 110, a storage circuitry 120, an input interface 130, a display 140, and a processing circuitry 150.

The NW interface 110 is connected to the processing circuitry 150, and controls transmission and communication of various types of data that are performed between the medical image processing apparatus 100a and a different apparatus. Examples of the different apparatus include a medical image storage apparatus such as a picture archiving and communication system (PACS) that stores medical image data, various modalities (medical imaging apparatuses), an electronic health record system, and the like, but the different apparatus is not limited to these. The NW interface 110 is implemented by a network card, a network adapter, a network interface controller (NIC), or the like.

The storage circuitry 120 prestores various types of information to be used in the processing circuitry 150. The storage circuitry 120 also stores various programs. The storage circuitry 120 is a nonvolatile storage device such as a hard disk drive (HDD), a solid state drive (SSD), or an integrated circuit storage device, for example, that stores various types of information. Alternatively, aside from the HDD, the SSD, and the like, the storage circuitry 120 may be a drive device that reads and writes various types of information from and into a portable storage medium such as a compact disc (CD), a digital versatile disc (DVD), or a flash memory, or a semiconductor memory device such as a random access memory (RAM). The storage circuitry 120 serves as an example of a storage unit.

The input interface 130 is implemented by a mouse, a keyboard, a drawing tablet in which a touch pen for receiving a user operation and a tablet are integrated, a trackball, a switch button, a touch pad on which an input operation is performed by touching an operation surface, a touch screen in which a display screen and a touch pad are integrated, a contactless input circuitry that uses an optical sensor, a voice input circuitry, and the like. The input interface 130 may include a plurality of devices for receiving operations performed by the user. The input interface 130 is connected to the processing circuitry 150, converts an input operation received from the user into an electric signal, and outputs the electric signal to the processing circuitry 150. In this specification, the input interface 130 is not limited to an input interface including a physical operational component such as a mouse or a keyboard. For example, an electric signal processing circuitry that receives an electric signal corresponding to an input operation, from an external input device provided separately from the apparatus, and outputs the electric signal to the processing circuitry 150 is also included in the examples of the input interface 130.

The display 140 displays various types of information under the control performed by the processing circuitry 150. For example, the display 140 outputs the generated candidate placement position display image 900, a graphical user interface (GUI) for receiving various operations from the user, and the like. Specifically, the display 140 is a liquid crystal display, a cathode ray tube (CRT) display, or the like. The input interface 130 and the display 140 may be integrated. For example, the input interface 130 and the display 140 may be implemented by a touch panel. The display 140 serves as an example of a display unit.

The processing circuitry 150 is a processor that implements a function corresponding to each program, by reading out a program from the storage circuitry 120 and executing the program. The processing circuitry 150 according to the present embodiment includes a receiving function 151, an acquisition function 152, a vessel diameter image generation function 153, a brain distance image generation function 154, a nerve fiber end point distance image generation function 155, an identification function 156, and a display control function 157. The receiving function 151 serves as an example of a receiving unit. The acquisition function 152 serves as an example of an acquisition unit. The vessel diameter image generation function 153 serves as an example of a vessel diameter image generation unit. The brain distance image generation function 154 and the nerve fiber end point distance image generation function 155 serve as an example of a distance image generation unit. Alternatively, the brain distance image generation function 154 may be an example of a brain distance image generation unit. In addition, the nerve fiber end point distance image generation function 155 may be an example of a nerve fiber end point distance image generation unit. The identification function 156 serves as an example of a candidate placement position identification unit. The display control function 157 serves as an example of a display control unit.

Here, processing functions of the receiving function 151, the acquisition function 152, the vessel diameter image generation function 153, the brain distance image generation function 154, the nerve fiber end point distance image generation function 155, the identification function 156, and the display control function 157, which are components of the processing circuitry 150, for example, are stored in the storage circuitry 120 in the form of programs executable by a computer. The processing circuitry 150 is a processor. For example, the processing circuitry 150 implements a function corresponding to each program, by reading out a program from the storage circuitry 120 and executing the program. In other words, the processing circuitry 150 in a state in which each program is read out has a corresponding function illustrated in the processing circuitry 150 in FIG. 2. The description has been given with reference to FIG. 2 assuming that processing functions to be performed by the receiving function 151, the acquisition function 152, the vessel diameter image generation function 153, the brain distance image generation function 154, the nerve fiber end point distance image generation function 155, the identification function 156, and the display control function 157 are implemented by a single processor, but the processing circuitry 150 may be formed by combining a plurality of independent processors, and a function may be implemented by each processor executing a program. In addition, the description has been given with reference to FIG. 2 assuming that a single storage circuitry 120 stores a program corresponding to each processing function, but a plurality of storage circuitries may be arranged in a dispersed manner, and the processing circuitry 150 may be configured to read out a corresponding program from an individual storage circuitry.

In the above description, an example in which a "processor" reads out a program corresponding to each function from a storage circuitry, and executes the program has been described, but the embodiment is not limited to this. The word "processor" means a circuitry such as, for example, a central processing unit (CPU), a graphics processing unit (GPU), an application specific integrated circuit (ASIC), or a programmable logic device (for example, simple programmable logic device (SPLD), complex programmable logic device (CPLD), and a field programmable gate array (FPGA)). In a case where a processor is a CPU, for example, the processor implements a function by reading out a program stored in a storage circuitry, and executing the program. On the other hand, in a case where a processor is an ASIC, in place of storing a program into the storage circuitry 120, a corresponding function is directly incorporated into a circuitry of the processor as a logic circuitry. Each processor according to the present embodiment is not limited to a case where each processor is formed as a single circuitry, and a plurality of independent circuitries may be combined into one processor to implement a corresponding function. Furthermore, a plurality of components in FIG. 2 may be integrated into one processor, and a corresponding function may be implemented.

The receiving function 151 receives various user operations via the input interface 130. For example, when a doctor or the like checks the candidate placement position display image 900 and determines a placement position of an intravascular electrode, the receiving function 151 receives an operation of determining the candidate placement position display image 900 that is performed by the doctor or the like. In addition, the receiving function 151 may receive a user operation of changing a candidate placement position on the candidate placement position display image 900. The receiving function 151 may receive a user operation of changing a prescribed standard for identifying a candidate placement position. The prescribed standard for identifying a candidate placement position will be described below. In addition, in a case where two or more activation regions 80 simultaneously exist in the brain of a subject, the receiving function 151 may receive a user operation of selecting either of the activation regions 80 that is to be measured.

In addition, the receiving function 151 may receive a user operation of selecting the type of a background image of the candidate placement position display image 900, and the type of character information to be displayed together with the candidate placement position display image 900. The type of the background image of the candidate placement position display image 900 and the character information to be displayed will be described below.

The acquisition function 152 acquires a medical image to be used for the generation of the candidate placement position display image 900, via the NW interface 110, for example. The acquisition source of the medical image may be a modality that has captured each medical image, or may be a medical image storage apparatus storing each medical image.

More specifically, the acquisition function 152 acquires the brain blood vessel image 910 and an image related to at least either one of a function region of the brain and the arrangement of a nerve in the brain. In the present embodiment, the acquisition function 152 acquires the brain function image 920 as an image related to a function region of the brain. In addition, the acquisition function 152 acquires the nerve fiber image 930 as an image related to the arrangement of a nerve in the brain.

The brain blood vessel image 910, the brain function image 920, and the nerve fiber image 930 that are to be acquired by the acquisition function 152 are images obtained by capturing images of the brain of the same subject.

The vessel diameter image generation function 153 generates a vessel diameter image from the brain blood vessel image 910. The vessel diameter image is an image indicating a size of a vessel diameter in the brain.

FIG. 3 is a diagram illustrating an example of the brain blood vessel image 910 and a vessel diameter image 911 according to the first embodiment. In the example illustrated in FIG. 3, in the brain blood vessel image 910, a size of a vessel diameter is indicated by a pixel value (contrasting density), for example. The brain blood vessel image 910 includes a blood vessel region and a background region that are distinguishable based on a pixel value. The vessel diameter image generation function 153 calculates a vessel diameter by obtaining a distance in a normal direction from a central line of a blood vessel region included in the brain blood vessel image 910, to an outer rim of the blood vessel region.

In addition, the vessel diameter image generation function 153 can obtain the central line of the blood vessel region by binarization processing and thinning processing of the brain blood vessel image 910. In addition, the vessel diameter image generation function 153 can obtain the outer rim of the blood vessel region by binarization processing and edge filter processing of the brain blood vessel image 910.

The vessel diameter image generation function 153 embeds a vessel diameter at a corresponding position (position of each pixel on the central line) into each pixel on the central line of the blood vessel region of the brain blood vessel image 910 as a pixel value. The vessel diameter image generation function 153 thereby generates the vessel diameter image 911 in which the thickness of a blood vessel at each position is indicated by a pixel value (contrasting density). For example, the vessel diameter image generation function 153 generates the vessel diameter image 911 in such a manner that a pixel value is smaller (color becomes darker) as a vessel diameter is thicker in the blood vessel region.

A vessel diameter image generation method is not limited to the above-described method, and a machine learning model trained using a data set of a brain blood vessel image prepared in advance and a corresponding vessel diameter image, for example, may be used. In this case, the vessel diameter image generation function 153 may input the brain blood vessel image 910 to the model, and obtain the vessel diameter image 911 as an output of the model.

Referring back to FIG. 2, the brain distance image generation function 154 generates a brain function distance image from the brain function image 920. The brain function distance image serves as an example of a distance image in the present embodiment. The brain function distance image is an image indicating a distance from an activation region at each pixel. The activation region serves as an example of a target position in the present embodiment.

FIG. 4 is a diagram illustrating an example of a brain function image 920a and a brain function distance image 921 according to the first embodiment. In the example illustrated in FIG. 4, one activation region 80 is visualized in the brain function image 920a. The brain distance image generation function 154 identifies the activation region 80 in the brain function image 920a. Then, the brain distance image generation function 154 generates the brain function distance image 921 in such a manner as to indicate a distance from the identified activation region in a brain tissue.

More specifically, the brain function image 920a is assumed to be an fMRI image, for example. In this case, the brain function image 920a has a pixel value indicating the intensity of a fixed difference in signal between a time with a task and a time without a task during the capturing of the fMRI image for a position where the difference is generated. Generally, because most locations of the brain region have the same signal at both of the time with a task and the time without a task, as illustrated in FIG. 4, the brain function image 920a is an image having a pixel value only in a partial region (activation region 80) of the brain. For this reason, the brain distance image generation function 154 can identify a region in the brain function image 920a that has a pixel value, as the activation region 80.

Then, the brain distance image generation function 154 generates the brain function distance image 921 by setting a distance from the activation region 80 to each pixel as a pixel value for all pixels of the brain function image 920a. That is, the brain function distance image 921 is an image indicating a distance from the activation region 80 to the position of each pixel as a pixel value (contrasting density).

In FIG. 4, the brain distance image generation function 154 sets a pixel value of each pixel corresponding to a brain region visualized in the brain function distance image 921, in such a manner that the pixel value becomes smaller (color becomes darker) as a distance from the activation region 80 gets farther. The brain distance image generation function 154 may set a pixel value in such a manner the pixel value becomes smaller (color becomes darker) as a distance from the activation region 80 gets closer.

In FIG. 4, broken lines are drawn like contour lines to indicate range with the same distances from the activation region 80 in the brain function distance image 921, but this illustration is representation in which a boundary of pixel values (contrasting density) is emphasized for the sake of explanation, and in reality, the broken lines need not be displayed.

In some cases, two or more activation regions 80 simultaneously exist in the brain of a subject. In a case where a plurality of activation regions 80 exist, the brain distance image generation function 154 generates the brain function distance image 921 for each of the activation regions 80.

FIG. 5 is a diagram illustrating an example of a brain function image 920b including two activation regions 80a and 80b, and brain function distance images 921a and 921b according to the first embodiment. The brain function distance image 921a (brain function distance image 1) is an image indicating a distance from the activation region 80a to each pixel. In addition, the brain function distance image 921b (brain function distance image 2) is an image indicating a distance from the activation region 80b to each pixel.

In a case where either one of the two activation regions 80a and 80b is a measurement target, the user may be enabled to select either one of the plurality of brain function distance images 921a and 921b before the generation of the candidate placement position display image 900.

The brain function images 920a and 920b illustrated in FIGS. 4 and 5 serve as an example of the brain function image 920. Hereinafter, in a case where there is no specific intention to limit the number of activation regions, the brain function images 920a and 920b will be simply referred to as the brain function images 920.

In addition, a generation method of the brain function distance image 921 is not limited to the above-described method. For example, the brain distance image generation function 154 may use a machine learning model trained using a data set of a brain function image including an activation region that has been prepared in advance and a corresponding brain function distance image. In this case, the brain distance image generation function 154 may input the brain function image 920 to the model, and obtain the brain function distance image 921 as an output of the model.

Referring back to FIG. 2, the nerve fiber end point distance image generation function 155 generates a nerve fiber end point distance image from the nerve fiber image 930. The nerve fiber end point distance image is another example of the distance image in the present embodiment. The nerve fiber end point distance image is an image indicating a distance from an end point of a nerve fiber to be measured. The end point of the nerve fiber to be measured is an end point of a nerve fiber in which a nerve activity in the brain is activated. The end point of the nerve fiber to be measured is rephrased as an end point of a nerve fiber corresponding to the activation region 80, among nerve fibers included in the brain. An end point of a nerve fiber that can be observed in a tractography of the MRI is assumed to be a point where cell bodies of nerve cells are densely arranged in terms of neurophysiology, and is important at a measurement target of a nerve activity by an intravascular electrode. The end point of the nerve fiber to be measured is another example of a target position in the present embodiment. An activation region identified from the brain function image 920, and an end point of a target nerve fiber identified from the nerve fiber image 930 are basically the same or close positions. That is, an occurrence position of a nerve activity to be measured can be identified based on whichever of an activation region identified from the brain function image 920, and an end point of a target nerve fiber identified from the nerve fiber image 930.

FIG. 6 is a diagram illustrating an example of the nerve fiber image 930 and a nerve fiber end point distance image 931 according to the first embodiment. The nerve fiber end point distance image generation function 155 generates the nerve fiber end point distance image 931 by setting distances from end points (nerve fiber end points) 71a and 71b of nerve fibers 70a and 70b to be measured, to corresponding pixels, as pixel values for all pixels of the nerve fiber image 930. That is, the nerve fiber end point distance image 931 is an image indicating distances from the nerve fiber end points 71a and 71b to the positions of corresponding pixels as pixel values (contrasting density).

As illustrated in FIG. 6, because the plurality of nerve fibers 70a and 70b are generally bundled, the nerve fiber end point distance image generation function 155 may collectively use one bundle of the nerve fiber end points 71a and 71b of the nerve fibers 70a and 70b included in one bundle, as a calculation reference point for distance.

In FIG. 6, the nerve fiber end point distance image generation function 155 sets pixel values of pixels corresponding to a brain region visualized in the nerve fiber end point distance image 931, in such a manner that pixel values become smaller (color becomes darker) as distances from the nerve fiber end points 71a and 71b get farther. Alternatively, the nerve fiber end point distance image generation function 155 may set pixel values in such a manner that pixel values become smaller (color becomes darker) as distances from the nerve fiber end points 71a and 71b get closer.

In FIG. 6, broken lines are illustrated to indicate ranges equidistant from the nerve fiber end points 71a and 71b in the nerve fiber end point distance image 931, but this illustration is representation in which a boundary of pixel values (contrasting density) is emphasized for the sake of explanation, and in reality, the broken lines need not be displayed. In addition, as described with reference to FIG. 5, in some cases, a plurality of regions in which a nerve activity in the brain is activated simultaneously exist. In this case, an end point of a nerve fiber belonging to a different bundle of nerve fibers existing at a distant position can become a measurement target. In this case, the nerve fiber end point distance image generation function 155 may generate the nerve fiber end point distance image 931 indicating a distance from a nerve fiber end point for each bundle of activated nerve fibers.

Referring back to FIG. 2, the identification function 156 identifies a position in a blood vessel region of the brain that satisfies a prescribed standard for both of a vessel diameter and a distance from a target position satisfying, based on the vessel diameter image 911, the brain function distance image 921, and the nerve fiber end point distance image 931, as a candidate placement position of a device.

The prescribed standard related to the vessel diameter is that the vessel diameter is equal to or larger than a prescribed size. The prescribed size of the vessel diameter depends on a diameter of a catheter to be used in placing a device. The diameter of the catheter varies depending on products, and is about 0.5 mm to 2 mm, for example. A diameter of a catheter planned to be used in a placement procedure of an intravascular electrode may be stored in the storage circuitry 120, for example. In this case, the prescribed size of the vessel diameter is a value obtained by adding a buffer value to the diameter of the catheter that is stored in the storage circuitry 120, for example. A value (for example, 0.3 mm or the like) of the buffer may be predefined and stored in the storage circuitry 120 or the like.

The prescribed standard related to the distance from the target position is that distances from the activation region 80 and the nerve fiber end points 71a and 71b to be measured are equal to or smaller than a prescribed distance. The prescribed distance is set to a distance at which an intravascular electrode can measure a signal attributed to a nerve activity, for example. In a case where a position with the smallest distance from the target position among blood vessels with vessel diameters equal to or larger than the prescribed size satisfies the prescribed standard related to the distance from the target position, the identification function 156 identifies the position with the smallest distance from the target position as a candidate placement position. In addition, in a case where there are two positions with the smallest distance from the target position, the identification function 156 may identify a plurality of candidate placement positions.

An identification method of a candidate placement position will be described in more detail. The identification function 156 performs position alignment of the vessel diameter image 911, the brain function distance image 921, and the nerve fiber end point distance image 931 for each pixel. A known technique can be employed as a method of alignment between images. As described above, a pixel value of the vessel diameter image 911 indicates a size of a vessel diameter. In addition, pixel values of the brain function distance image 921 and the nerve fiber end point distance image 931 indicate distances from the target position. For this reason, the identification function 156 can identify a vessel diameter at each pixel and a distance from the target position based on pixel values of the vessel diameter image 911, pixel values of the brain function distance image 921, and pixel values of the nerve fiber end point distance image 931. In a case where a distance from the target position to a pixel with the smallest distance from the target position among pixels with vessel diameters equal to or larger than the prescribed size is equal to or smaller than the prescribed distance, the identification function 156 identifies the position of the pixel as a candidate placement position.

The display control function 157 displays the candidate placement position identified by the identification function 156, in an intensified manner on an image indicating the brain of a subject. An image in which a candidate placement position is displayed by being superimposed on an image indicating the brain of a subject will be referred to as the candidate placement position display image 900. The display control function 157 displays the candidate placement position display image 900 on the display 140.

An image indicating the brain of a subject that is be used in the candidate placement position display image 900 is, for example, the brain blood vessel image 910, the brain function image 920, the nerve fiber image 930, the vessel diameter image 911, the brain function distance image 921, or the nerve fiber end point distance image 931, but is not limited to these.

In addition, the display control function 157 may display characters indicating a vessel diameter at the candidate placement position or a distance from the target position, on the image indicating the brain.

FIGS. 7 to 10 illustrate variations of display modes of the candidate placement position display image 900.

FIG. 7 is a diagram illustrating an example of a candidate placement position display image 900a in which the brain blood vessel image 910 is used according to the first embodiment. In the candidate placement position display image 900a, a graphic 60 indicating a candidate placement position is displayed by being superimposed on the brain blood vessel image 910. By such display, a doctor or the like, which is a user, can recognize a location of the candidate placement position in the brain of the subject, and a route of a blood vessel reaching the candidate placement position.

FIG. 8 is a diagram illustrating an example of a candidate placement position display image 900b in which the vessel diameter image 911 is used according to the first embodiment. In the candidate placement position display image 900b, the FIG. 60 indicating a candidate placement position is displayed by being superimposed on the vessel diameter image 911. By such display, a doctor or the like, which is a user, can easily recognize a location of the candidate placement position in the brain of the subject and a vessel diameter of a blood vessel in the brain that includes a blood vessel reaching the candidate placement position.

In addition, in FIG. 8, the display control function 157 displays characters "vessel diameter: 4 mm" indicating a vessel diameter at the candidate placement position, on the candidate placement position display image 900b. The display control function 157 displays the characters in a display field 61a provided at a position not overlapping the FIG. 60 indicating the candidate placement position on the vessel diameter image 911, for example.

FIG. 9 is a diagram illustrating an example of a candidate placement position display image 900c in which the brain function image 920 is used according to the first embodiment. In the candidate placement position display image 900c, the FIG. 60 indicating a candidate placement position is displayed by being superimposed on the brain function image 920b. On the brain function image 920b in FIG. 9, the two activation regions 80a and 80b exist. In the example illustrated in FIG. 9, the activation region 80a is selected by the user as a measurement target out of the two activation regions 80a and 80b. In this case, the display control function 157 displays the FIG. 60 at a position with the smallest distance from the activation region 80a in a blood vessel region in which vessel diameters are equal to or larger than the prescribed size. In a case where the user selects the activation region 80b, the display control function 157 displays the FIG. 60 at a position with the smallest distance from the activation region 80b in a blood vessel region in which vessel diameters are equal to or larger than the prescribed size.

In addition, in FIG. 9, the display control function 157 displays characters "distance: 4 mm" indicating a distance from the target position to the candidate placement position, in a display field 61b on the candidate placement position display image 900c. By the display, the user can recognize that a distance between a target position (activation region) and an intravascular electrode becomes 4 mm in a case where an intravascular electrode is placed at a candidate placement position indicated by the FIG. 60.

FIG. 10 is a diagram illustrating an example of a candidate placement position display image 900d in which the nerve fiber image 930 is used according to the first embodiment. In the candidate placement position display image 900d, the FIG. 60 indicating a candidate placement position is displayed by being superimposed on the nerve fiber image 930.

In addition, in FIG. 10, the display control function 157 displays characters "distance: 6 mm" indicating a distance from the target position to the candidate placement position, in a display field 61c on the candidate placement position display image 900d.

An image to be used in the candidate placement position display image 900, and a display mode of the candidate placement position display image 900 may be predefined, or may be made selectable or changeable by a user operation. For example, the display control function 157 may display, as a background image of the candidate placement position display image 900, a selection screen on which the user can select which of the brain blood vessel image 910, the brain function image 920, the nerve fiber image 930, the vessel diameter image 911, the brain function distance image 921, and the nerve fiber end point distance image 931 is to be used. In addition, whether to display character information indicating a vessel diameter at the candidate placement position, a distance from the target position to the candidate placement position, or the like, together with the candidate placement position display image 900 as in FIGS. 8 to 10 may be made selectable or changeable by a user operation.

In addition, the display control function 157 may display a list box or the like by which the type of a background image of the candidate placement position display image 900, and the type of character information to be displayed can be switched, outside a field of the candidate placement position display image 900 on the display 140.

In addition, the display control function 157 displays a determination button via which an operation performed by a user who determines a placement position can be received, for example, on the display 140 together with the candidate placement position display image 900. By the user pressing the determination button, a candidate placement position indicated by the FIG. 60 on the candidate placement position display image 900 is determined as a placement position of an intravascular electrode.

Here, a flow of identification processing of a candidate placement position of an intravascular electrode that is to be executed by the medical image processing apparatus 100a having the above-described configuration will be described.

FIG. 11 is a flowchart illustrating an example of a flow of identification processing of a candidate placement position of an intravascular electrode according to the first embodiment.

First of all, in step S1, the acquisition function 152 acquires the brain blood vessel image 910, the brain function image 920, and the nerve fiber image 930 obtained by capturing images of a subject.

Then, in step S2, the vessel diameter image generation function 153 generates the vessel diameter image 911 from the brain blood vessel image 910.

In addition, in step S3, the brain distance image generation function 154 generates the brain function distance image 921 from the brain function image 920.

In addition, in step S4, the nerve fiber end point distance image generation function 155 generates the nerve fiber end point distance image 931 from the nerve fiber image 930.

Then, in step S5, the identification function 156 identifies a candidate placement position of an intravascular electrode in a blood vessel region of a brain of the subject based on the vessel diameter image 911, the brain function distance image 921, and the nerve fiber end point distance image 931. For example, the identification function 156 performs position alignment of the vessel diameter image 911, the brain function distance image 921, and the nerve fiber end point distance image 931 for each pixel, and identifies a position of a pixel with the smallest distance from the target position among pixels with vessel diameters equal to or larger than a prescribed size, as a candidate placement position. In a case where two or more activation regions 80 simultaneously exist in the brain of the subject, the user may be enabled to select which of the plurality of activation regions 80 is to be measured, before the processing in step S5.

Then, in step S6, the display control function 157 displays the candidate placement position display image 900 in which the FIG. 60 indicating the candidate placement position identified by the identification function 156 is superimposed on an image indicating the brain of the subject (for example, the brain blood vessel image 910, the brain function image 920, the nerve fiber image 930, the vessel diameter image 911, the brain function distance image 921, or the nerve fiber end point distance image 931), on the display 140. In addition, the display control function 157 displays a determination button via which an operation performed by a user who determines a placement position can be received, for example, on the display 140 together with the candidate placement position display image 900.

In step S7, the receiving function 151 receives an operation of determining a placement position that is performed by the user who has checked the candidate placement position display image 900 displayed on the display 140. For example, the receiving function 151 may receive a user operation of pressing the determination button on the display 140. In a case where the determination button is pressed by the user, the candidate placement position displayed on the candidate placement position display image 900 is stored into the storage circuitry 120 as a placement position. In addition, the determined placement position may be transmitted to another apparatus via the NW interface 110. Examples of the other apparatus includes a modality such as a blood vessel imaging apparatus that is to be used in intervention treatment for placing an intravascular electrode.

In addition, the receiving function 151 may receive a user operation of changing a candidate placement position, on the candidate placement position display image 900. For example, in a case where the user performs an operation of changing the position of the FIG. 60 on the candidate placement position display image 900, the receiving function 151 may receive the changed position of the FIG. 60 as a placement position. By the placement position being determined in the processing in step S7, the processing in this flowchart ends.

In this manner, the medical image processing apparatus 100a according to the present embodiment generates the vessel diameter image 911 indicating a size of a vessel diameter in the brain, from the brain blood vessel image 910, also identifies a target position corresponding to a nerve activity, which is a measurement target in the brain, based on the brain function image 920 and the nerve fiber image 930, and generates the brain function distance image 921 and the nerve fiber end point distance image 931 indicating a distance from the identified target position. The medical image processing apparatus 100a according to the present embodiment identifies a position with both of a vessel diameter and a distance from the target position satisfying a prescribed standard on the blood vessel, based on the generated vessel diameter image 911, the brain function distance image 921, and the nerve fiber end point distance image 931, as a candidate placement position of an intravascular electrode, and displays the candidate placement position in an intensified manner in an image indicating the brain (for example, the brain blood vessel image 910, the brain function image 920, the nerve fiber image 930, the vessel diameter image 911, the brain function distance image 921, or the nerve fiber end point distance image 931). For this reason, by using the medical image processing apparatus 100a according to the present embodiment, the user can easily recognize a candidate placement position where an intravascular electrode can be placed near an occurrence location of a measurement target nerve activity in the brain. For this reason, the user can easily place an intravascular electrode at an ideal location for measurement of a nerve activity, and suppress a decline in measurement sensitivity.

In addition, the medical image processing apparatus 100a according to the present embodiment generates, as a distance image for identifying a target position corresponding to a nerve activity, which is a measurement target in the brain, the brain function distance image 921 indicating a distance from an activation region of a nerve activity in the brain and the nerve fiber end point distance image 931 indicating a distance from an end point of a measurement target nerve fiber in the brain. In addition, in the present embodiment, the brain blood vessel image 910 to be used for the generation of the vessel diameter image 911, the brain function image 920 to be used for the generation of the brain function distance image 921, and the nerve fiber image 930 to be used for the generation of the nerve fiber end point distance image 931 are images obtained by capturing images of the brain of the same subject. For this reason, by using the medical image processing apparatus 100a according to the present embodiment, it is possible to precisely identify a target position based on two types of distance images generated from images obtained by capturing images of the brain of the same subject. In addition, because all images used for the identification of a candidate placement position are images obtained by capturing images of the brain of the same subject, the precision of position alignment between images for identification of a vessel diameter and a distance from the target position becomes higher, and it is possible to precisely identify a candidate placement position suitable for measurement.

In addition, the vessel diameter image 911 according to the present embodiment is an image indicating a size of a vessel diameter in the brain as a pixel value, and the brain function distance image 921 and the nerve fiber end point distance image 931 are images indicating a distance from the target position as a pixel value. The medical image processing apparatus 100a according to the present embodiment identifies, based on pixel values of the vessel diameter image 911, pixel values of the brain function distance image 921, and pixel values of the nerve fiber end point distance image 931, a position of a pixel with the smallest distance from the target position among pixels with vessel diameters equal to or larger than a prescribed size, as a candidate placement position. For this reason, by using to the medical image processing apparatus 100a according to the present embodiment, it is possible to precisely identify a candidate placement position of an intravascular electrode that is suitable for the measurement of a target nerve activity, for each pixel.

In addition, the medical image processing apparatus 100a according to the present embodiment displays a character indicating a vessel diameter at a candidate placement position or a distance from the target position, on an image indicating a brain. For this reason, by using the medical image processing apparatus 100a according to the present embodiment, the user can easily recognize a vessel diameter at a candidate placement position displayed in an intensified manner, or a distance between the target position and an intravascular electrode in a case where the intravascular electrode is placed. With this configuration, the user can study a placement position simultaneously considering a vessel diameter at the candidate placement position or a distance from the target position that is indicated by character information, while visually checking the candidate placement position.

### Second Embodiment

FIG. 12 is a diagram illustrating the overview of input-output of processing of a medical image processing apparatus according to the second embodiment. In the above-described first embodiment, the medical image processing apparatus 100a outputs the candidate placement position display image 900 using the brain blood vessel image 910, the brain function image 920, and the nerve fiber image 930 as inputs. Nevertheless, a medical image processing apparatus needs not always use both of the brain function image 920 and the nerve fiber image 930, and may identify a target position corresponding to a nerve activity, which is a measurement target in the brain, based on an image related to at least either one of the brain function image 920 and the nerve fiber image 930. Specifically, in the second embodiment, a medical image processing apparatus outputs the candidate placement position display image 900 using the brain blood vessel image 910 and the brain function image 920 as inputs.

Similarly to the first embodiment, the brain blood vessel image 910 is an image in which a blood vessel of a brain of a subject is visualized. In addition, similarly to the first embodiment, the brain function image 920 is an image such as an fMRI image in which an activation region of a nerve activity desired to be measured by an intravascular electrode is visualized.

FIG. 13 is a diagram illustrating an example of a configuration of a medical image processing apparatus 100b according to the second embodiment. Similarly to the medical image processing apparatus 100a according to the first embodiment that is illustrated in FIG. 2, the medical image processing apparatus 100b includes the NW interface 110, the storage circuitry 120, the input interface 130, the display 140, and the processing circuitry 150.

In addition, the processing circuitry 150 includes the receiving function 151, an acquisition function 152a, the vessel diameter image generation function 153, the brain distance image generation function 154, an identification function 156a, and the display control function 157.

The receiving function 151, the vessel diameter image generation function 153, the brain distance image generation function 154, and the display control function 157 have functions similar to those in the first embodiment.

For example, the vessel diameter image generation function 153 generates the vessel diameter image 911 from the brain blood vessel image 910 similarly to the first embodiment.

In addition, the brain distance image generation function 154 generates the brain function distance image 921 from the brain function image 920 similarly to the first embodiment. The brain function distance image 921 serves as an example of a distance image in the present embodiment.

In addition, the display control function 157 displays the candidate placement position display image 900 in which a candidate placement position is displayed in an intensified manner on an image indicating the brain of the subject, on the display 140 similarly to the first embodiment. In the present embodiment, an image indicating the brain of the subject that is used in the candidate placement position display image 900 is, for example, the brain blood vessel image 910, the brain function image 920, the vessel diameter image 911, or the brain function distance image 921.

The acquisition function 152a acquires the brain blood vessel image 910 and the brain function image 920 via the NW interface 110, for example.

The brain blood vessel image 910 and the brain function image 920 that are to be acquired by the acquisition function 152a are images obtained by capturing images of the brain of the same subject.

Unlike the identification function 156 according to the first embodiment, the identification function 156a identifies a candidate placement position without using the nerve fiber end point distance image 931. Apart from this point, the identification function 156a has a function similar to that of the identification function 156 according to the first embodiment.

More specifically, the identification function 156a identifies a candidate placement position of an intravascular electrode based on the vessel diameter image 911 and the brain function distance image 921. The identification function 156a according to the present embodiment performs position alignment of the vessel diameter image 911 and the brain function distance image 921 for each pixel, and identifies a vessel diameter at each pixel and a distance from the target position based on pixel values of the vessel diameter image 911 and pixel values of the brain function distance image 921. Then, in a case where a distance from the target position to a pixel with the smallest distance from the target position among pixels with vessel diameters equal to or larger than the prescribed size is equal to or smaller than the prescribed distance, the identification function 156a identifies the position of the pixel as a candidate placement position.

Here, a flow of identification processing of a candidate placement position of an intravascular electrode that is to be executed by the medical image processing apparatus 100b according to the present embodiment that has the above-described configuration will be described. FIG. 14 is a flowchart illustrating an example of a flow of identification processing of a candidate placement position of an intravascular electrode according to the second embodiment.

First of all, in step S11, the acquisition function 152a acquires the brain blood vessel image 910 and the brain function image 920 obtained by capturing images of a subject.

Then, in step S12, the vessel diameter image generation function 153 generates the vessel diameter image 911 from the brain blood vessel image 910.

In addition, in step S13, the brain distance image generation function 154 generates the brain function distance image 921 from the brain function image 920.

Then, in step S14, the identification function 156a identifies a candidate placement position of an intravascular electrode in a blood vessel region of a brain of the subject based on the vessel diameter image 911 and the brain function distance image 921.

Then, in step S15, the display control function 157 displays the candidate placement position display image 900 in which the FIG. 60 indicating the candidate placement position identified by the identification function 156a is superimposed on an image indicating the brain of the subject (for example, the brain blood vessel image 910, the brain function image 920, the vessel diameter image 911, or the brain function distance image 921), on the display 140.

Then, in step S16, the receiving function 151 receives an operation of determining a placement position that is performed by the user who has checked the candidate placement position display image 900 displayed on the display 140. The processing to be performed after the placement position is determined is similar to that in the first embodiment. Here, the processing in this flowchart ends.

In this manner, the medical image processing apparatus 100b according to the present embodiment generates the brain function distance image 921 indicating a distance from an activation region of a nerve activity in the brain, from the brain function image 920, and identifies a candidate placement position of an intravascular electrode based on the brain blood vessel image 910 and the brain function distance image 921. For this reason, by using the medical image processing apparatus 100b according to the present embodiment, in addition to an effect similar to that of the first embodiment, even in a case where it is difficult to obtain the nerve fiber image 930, it is possible to identify a candidate placement position of an intravascular electrode.

In addition, in the present embodiment, the brain blood vessel image 910 to be used for the generation of the vessel diameter image 911 and the brain function image 920 to be used for the generation of the brain function distance image 921 are images obtained by capturing the brain of the same subject. For this reason, according to the medical image processing apparatus 100b according to the present embodiment, similarly to the first embodiment, by precisely executing position alignment between images for identification of a vessel diameter and a distance from the target position, it is possible to precisely identify a candidate placement position suitable for measurement.

### Third Embodiment

FIG. 15 is a diagram illustrating the overview of input-output of processing of a medical image processing apparatus according to the third embodiment. In the above-described first embodiment, the medical image processing apparatus 100a outputs the candidate placement position display image 900 using the brain blood vessel image 910, the brain function image 920, and the nerve fiber image 930 as inputs. In contract to this, in the third embodiment, a medical image processing apparatus outputs the candidate placement position display image 900 using the brain blood vessel image 910 and the nerve fiber image 930 as inputs.

Similarly to the first embodiment, the brain blood vessel image 910 is an image in which a blood vessel of a brain of a subject is visualized. In addition, similarly to the first embodiment, the nerve fiber image 930 is an image such as a tractography of the MRI in which a nerve fiber in the brain is visualized.

FIG. 16 is a diagram illustrating an example of a configuration of a medical image processing apparatus 100c according to the third embodiment. Similarly to the medical image processing apparatus 100a according to the first embodiment that is illustrated in FIG. 2, the medical image processing apparatus 100c includes the NW interface 110, the storage circuitry 120, the input interface 130, the display 140, and the processing circuitry 150.

In addition, the processing circuitry 150 includes the receiving function 151, an acquisition function 152b, the vessel diameter image generation function 153, the nerve fiber end point distance image generation function 155, an identification function 156b, and the display control function 157.

The receiving function 151, the vessel diameter image generation function 153, the nerve fiber end point distance image generation function 155, and the display control function 157 have functions similar to those in the first embodiment.

For example, the vessel diameter image generation function 153 generates the vessel diameter image 911 from the brain blood vessel image 910 similarly to the first embodiment.

In addition, the nerve fiber end point distance image generation function 155 generates the nerve fiber end point distance image 931 from the nerve fiber image 930 similarly to the first embodiment. The nerve fiber end point distance image 931 is an example of a distance image in the present embodiment.

In addition, the display control function 157 displays the candidate placement position display image 900 in which a candidate placement position is displayed in an intensified manner on an image indicating the brain of the subject, on the display 140 similarly to the first embodiment. In the present embodiment, an image indicating the brain of the subject that is used in the candidate placement position display image 900 is, for example, the brain blood vessel image 910, the nerve fiber image 930, the vessel diameter image 911, or the nerve fiber end point distance image 931.

The acquisition function 152b acquires the brain blood vessel image 910 and the nerve fiber image 930 via the NW interface 110, for example.

The brain blood vessel image 910 and the nerve fiber image 930 that are to be acquired by the acquisition function 152b are images obtained by capturing images of the brain of the same subject.

Unlike the identification function 156 according to the first embodiment, the identification function 156b identifies a candidate placement position without using the brain function distance image 921. Apart from this point, the identification function 156b has a function similar to that of the identification function 156 according to the first embodiment.

More specifically, the identification function 156b identifies a candidate placement position of an intravascular electrode based on the vessel diameter image 911 and the nerve fiber end point distance image 931. The identification function 156b according to the present embodiment performs position alignment of the vessel diameter image 911 and the nerve fiber end point distance image 931 for each pixel, and identifies a vessel diameter at each pixel and a distance from the target position based on pixel values of the vessel diameter image 911 and pixel values of the nerve fiber end point distance image 931. Then, in a case where a distance from the target position to a pixel with the smallest distance from the target position among pixels with vessel diameters equal to or larger than the prescribed size is equal to or smaller than the prescribed distance, the identification function 156b identifies the position of the pixel as a candidate placement position.

Here, a flow of identification processing of a candidate placement position of an intravascular electrode that is to be executed by the medical image processing apparatus 100c according to the present embodiment that has the above-described configuration will be described. FIG. 17 is a flowchart illustrating an example of a flow of identification processing of a candidate placement position of an intravascular electrode according to the third embodiment.

First of all, in step S21, the acquisition function 152b acquires the brain blood vessel image 910 and the nerve fiber image 930 obtained by capturing images of a subject.

Then, in step S22, the vessel diameter image generation function 153 generates the vessel diameter image 911 from the brain blood vessel image 910.

In addition, in step S23, the nerve fiber end point distance image generation function 155 generates the nerve fiber end point distance image 931 from the nerve fiber image 930.

Then, in step S24, the identification function 156b identifies a candidate placement position of an intravascular electrode in a blood vessel region of a brain of the subject based on the vessel diameter image 911 and the nerve fiber end point distance image 931.

Then, in step S25, the display control function 157 displays the candidate placement position display image 900 in which the FIG. 60 indicating the candidate placement position identified by the identification function 156b is superimposed on an image indicating the brain of the subject (for example, the brain blood vessel image 910, the nerve fiber image 930, the vessel diameter image 911, or the nerve fiber end point distance image 931), on the display 140.

Then, in step S26, the receiving function 151 receives an operation of determining a placement position that is performed by the user who has checked the candidate placement position display image 900 displayed on the display 140. The processing to be performed after the placement position is determined is similar to that in the first embodiment. Here, the processing in this flowchart ends.

In this manner, the medical image processing apparatus 100c according to the present embodiment generates the nerve fiber end point distance image 931 indicating a distance from an end point of a measurement target nerve fiber, from the nerve fiber image 930, and identifies a candidate placement position of an intravascular electrode based on the brain blood vessel image 910 and the nerve fiber end point distance image 931. For this reason, according to the medical image processing apparatus 100c according to the present embodiment, in addition to an effect similar to that of the first embodiment, even in a case where it is difficult to obtain the brain function image 920, it is possible to identify a candidate placement position of an intravascular electrode.

In addition, in the present embodiment, the brain blood vessel image 910 to be used for the generation of the vessel diameter image 911 and the nerve fiber image 930 to be used for the generation of the nerve fiber end point distance image 931 are images obtained by capturing the brain of the same subject. For this reason, according to the medical image processing apparatus 100c according to the present embodiment, similarly to the first embodiment, by precisely executing position alignment between images for identification of a vessel diameter and a distance from the target position, it is possible to precisely identify a candidate placement position suitable for measurement.

### Fourth Embodiment

FIG. 18 is a diagram illustrating the overview of input-output of processing of a medical image processing apparatus according to the fourth embodiment. In the above-described first embodiment, the medical image processing apparatus 100a outputs the candidate placement position display image 900 using the brain blood vessel image 910, the brain function image 920, and the nerve fiber image 930 as inputs. In contract to this, in the fourth embodiment, a medical image processing apparatus outputs the candidate placement position display image 900 using the brain blood vessel image 910 and a brain region atlas 940 as inputs.

Similarly to the first embodiment, the brain blood vessel image 910 is an image in which a blood vessel of a brain of a subject is visualized.

The brain region atlas 940 is information indicating anatomical arrangement of a brain region involving a nerve activity. More specifically, in the brain region atlas 940, a function of a brain and a three-dimensional coordinate of a region having the function are associated. The brain region atlas 940 will also be referred to as a brain atlas, a brain map, and brain function mapping. The brain region atlas 940 is not information unique to a subject, but information common to brains of general human bodies. The brain region atlas 940 serves as an example of an image related to a function region of a brain in the present embodiment.

FIG. 19 is a diagram illustrating an example of a configuration of a medical image processing apparatus 100d according to the fourth embodiment. Similarly to the medical image processing apparatus 100a according to the first embodiment that is illustrated in FIG. 2, the medical image processing apparatus 100d includes the NW interface 110, the storage circuitry 120, the input interface 130, the display 140, and the processing circuitry 150.

In addition, the processing circuitry 150 includes the receiving function 151, an acquisition function 152c, the vessel diameter image generation function 153, an identification function 156c, the display control function 157, and a brain region distance image generation function 158. The brain region distance image generation function 158 is an example of a distance image generation unit in the present embodiment.

The receiving function 151, the vessel diameter image generation function 153, and the display control function 157 have functions similar to those in the first embodiment.

For example, the vessel diameter image generation function 153 generates the vessel diameter image 911 from the brain blood vessel image 910 similarly to the first embodiment.

In addition, the display control function 157 displays the candidate placement position display image 900 in which a candidate placement position is displayed in an intensified manner on an image indicating the brain of the subject, on the display 140 similarly to the first embodiment. In the present embodiment, an image indicating the brain of the subject that is used in the candidate placement position display image 900 is, for example, the brain blood vessel image 910 or the vessel diameter image 911.

The acquisition function 152c acquires the brain blood vessel image 910 and the brain region atlas 940 via the NW interface 110, for example. An acquisition source of the brain region atlas 940 is not specifically limited, and the brain region atlas 940 may be information or the like on the internet, for example. In addition, the brain region atlas 940 may be prestored in the storage circuitry 120. In this case, the acquisition function 152c reading out the brain region atlas 940 from the storage circuitry 120 is also assumed to be an example of acquisition.

The brain region distance image generation function 158 generates a brain region distance image from the brain region atlas 940. The brain region distance image is an example of a distance image in the present embodiment.

FIG. 20 is a diagram illustrating an example of the brain region atlas 940 and a brain region distance image 941 according to the fourth embodiment.

The brain region distance image 941 is an image indicating a distance from a brain region from which a nerve activity is to be measured. The brain region distance image generation function 158 generates the brain region distance image 941 by setting distances to respective pixels from a brain region from which a nerve activity is to be measured, as pixel values for all pixels, for example. That is, the brain region distance image 941 is an image indicating a distance to the position of each pixel from a brain region from which a nerve activity is to be measured, as a pixel value (contrasting density).

A brain region from which a nerve activity is to be measured is an example of a target position in the present embodiment. The brain region from which a nerve activity is to be measured is selected by the user, for example. The display control function 157 may display a selection screen on which a user operation of designating a measurement target brain region on the brain region atlas 940 can be received, on the display 140. In addition, the receiving function 151 may receive a user operation of designating a measurement target brain region.

In FIG. 20, broken lines are illustrated to indicate ranges with the same distances from a brain region in the brain region distance image 941, from which a nerve activity is to be measured, but this illustration is representation in which a boundary of pixel values (contrasting density) is emphasized for the sake of explanation, and in reality, the broken lines need not be displayed.

Unlike the identification function 156 according to the first embodiment, the identification function 156c identifies a candidate placement position of an intravascular electrode based on the vessel diameter image 911 and the brain region distance image 941. The identification function 156c according to the present embodiment performs position alignment of the vessel diameter image 911 and the brain region distance image 941 for each pixel, and identifies a vessel diameter at each pixel and a distance from the target position based on pixel values of the vessel diameter image 911 and pixel values of the brain region distance image 941. Then, in a case where a distance from the target position to a pixel with the smallest distance from the target position among pixels with vessel diameters equal to or larger than the prescribed size is equal to or smaller than the prescribed distance, the identification function 156c identifies the position of the pixel as a candidate placement position. Because the brain region atlas 940 is not adapted to an individual subject, but is a model assumed for a general human body, the identification function 156c may correct the brain region distance image 941 in accordance with the vessel diameter image 911 at the time of position alignment of the vessel diameter image 911 and the brain region distance image 941.

Here, a flow of identification processing of a candidate placement position of an intravascular electrode that is to be executed by the medical image processing apparatus 100d according to the present embodiment that has the above-described configuration will be described. FIG. 21 is a flowchart illustrating an example of a flow of identification processing of a candidate placement position of an intravascular electrode according to the fourth embodiment.

First of all, in step S31, the acquisition function 152c acquires the brain blood vessel image 910 and the brain region atlas 940.

Then, in step S32, the vessel diameter image generation function 153 generates the vessel diameter image 911 from the brain blood vessel image 910.

In addition, in step S33, the brain region distance image generation function 158 generates the brain region distance image 941 from the brain region atlas 940.

Then, in step S34, the identification function 156c identifies a candidate placement position of an intravascular electrode in a blood vessel region of a brain of the subject based on the vessel diameter image 911 and the brain region distance image 941.

Then, in step S35, the display control function 157 displays the candidate placement position display image 900 in which the FIG. 60 indicating the candidate placement position identified by the identification function 156c is superimposed on an image indicating the brain of the subject (for example, the brain blood vessel image 910 or the vessel diameter image 911), on the display 140.

Then, in step S36, the receiving function 151 receives an operation of determining a placement position that is performed by the user who has checked the candidate placement position display image 900 displayed on the display 140. The processing to be performed after the placement position is determined is similar to that in the first embodiment. Here, the processing in this flowchart ends.

In this manner, the medical image processing apparatus 100d according to the present embodiment identifies a candidate placement position of an intravascular electrode based on the vessel diameter image 911 generated from the brain blood vessel image 910 and the brain region distance image 941 generated from the brain region atlas 940. For this reason, by using the medical image processing apparatus 100d according to the present embodiment, in addition to an effect similar to that of the first embodiment, even in a case where it is difficult to obtain the brain function image 920 and the nerve fiber image 930, it is possible to identify a candidate placement position of an intravascular electrode.

For example, in some cases, it is difficult to execute image capturing by the MRI for a reason such as a magnetic device placed in a body of a subject, an artificial joint used by a subject, or a magnetic foreign object existing in a brain of a subject due to a past accident. Because the medical image processing apparatus 100d according to the present embodiment does not use the brain function image 920 and the nerve fiber image 930, by employing the CT angiography or the DSA as the brain blood vessel image 910, for example, it is possible to identify a candidate placement position of an intravascular electrode without executing image capturing by the MRI. For this reason, even in a situation where it is difficult to apply the first to third embodiments, it is possible to apply the fourth embodiment.

### Modified Example 1

In the above-described first to fourth embodiments, in a case where a distance from the target position to a pixel with the smallest distance from the target position among pixels with vessel diameters equal to or larger than the prescribed measurement is equal to or smaller than the prescribed distance, the identification functions 156 and 156a to 156c identify the position of the pixel as a candidate placement position. Nevertheless, pixels with vessel diameters equal to or larger than the prescribed measurement do not always include a pixel with a distance from the target position that is equal to or smaller than the prescribed distance.

For example, in a case where a position with both of a vessel diameter and a distance from the target position satisfying a prescribed standard on the blood vessel does not exist, the identification functions 156 and 156a to 156c may identify a position with either one of a vessel diameter and a distance from the target position satisfying a prescribed standard, as an alternative candidate. For example, in addition, in a case where a pixel with a vessel diameter equal to or larger than the prescribed measurement does not fall within a range in which a distance from the target position is equal to or smaller than the prescribed distance, the identification function 156 may identify a position of a pixel with a vessel diameter equal to or larger than the prescribed measurement among pixels contiguous with a pixel with the smallest distance from the target position as a candidate placement position.

In a case where pixels with vessel diameters equal to or larger than the prescribed measurement do not include a pixel with a distance from the target position that is equal to or smaller than the prescribed distance, the identification functions 156 and 156a to 156c may identify a position of a pixel corresponding to a case where either or both of a vessel diameter and a distance from the target position in the prescribed standard is changed. For example, the identification functions 156 and 156a to 156c may identify "by how many millimeters a vessel diameter is to be increased from the current prescribed standard in order to include a pixel with a distance from the target position that is equal to or smaller than the prescribed distance", or "by how many millimeters an upper limit distance from the target position is to be increased from the current prescribed distance in order include a pixel with a vessel diameter satisfying the current prescribed standard".

In addition, in a case where pixels with vessel diameters equal to or larger than the prescribed size do not include a pixel with a distance from the target position that is equal to or smaller than the prescribed distance, the identification functions 156 and 156a to 156c may output a result indicating that "a candidate placement position is not to be identified", for example. In this case, the display control function 157 may display a message indicating that "a blood vessel satisfying a prescribed standard does not exist (cannot be identified)", on the display 140 in place of the candidate placement position display image 900.

In addition, the display control function 157 may display the identified alternative candidate and a suggestion regarding a change in a prescribed standard that is based on a vessel diameter at the position of the alternative candidate and a distance from the target position, on the display 140. For example, the display control function 157 may display character information indicating a suggestion that "among positions satisfying a currently-designated vessel diameter (or diameter of a catheter), a distance closest to the target position is X mm" or "among blood vessels satisfying a currently-designated distance from the target position, a diameter of the thickest blood vessel is Y mm", on the display 140.

In addition, in a case where a position with both of a vessel diameter and a distance from the target position satisfying a prescribed standard on the blood vessel does not exist, the receiving function 151 may receive a user operation of changing the prescribed standard. In this case, the identification functions 156 and 156a to 156c identify a candidate placement position again based on the changed prescribed standard. In addition, the display control function 157 displays the candidate placement position display image 900 in which a candidate placement position identified based on the changed prescribed standard is displayed in an intensified manner, on the display 140.

By such display of the alternative candidate and a suggestion related to a change in prescribed standard, the user can appropriately change the prescribed standard with reference to the displayed alternative candidate and suggestion. For example, because the user cannot recognize how to change a prescribed standard to cause a corresponding pixel to exist, by only simply indicating that a position satisfying the prescribed standard is not identified, there is a possibility that reviewing content to be changed may take time or that the standards may be eased more than necessary. By display of the alternative candidate and a suggestion related to changing the prescribed standard, it is possible to support a work of the user, and contribute to streamlining of candidate placement position identification.

### Modified Example 2

In addition, in the above-described first embodiment, in a case where there are two or more positions with the smallest distance from the target position, the identification function 156 may identify a plurality of candidate placement positions. In a case where a plurality of candidate placement positions satisfying a prescribed standard exit in this manner, the display control function 157 may number the plurality of candidate placement positions in descending order of vessel diameter, or in ascending order of a distance from the target position, and display the plurality of candidate placement positions on the candidate placement position display image 900.

By displaying a plurality of candidate placement positions while executing prioritization in accordance with a vessel diameter or a distance from the target position in this manner, it is possible to support appropriate determination of intent when the user determines a placement position.

### Modified Example 3

In the above-described first to the fourth embodiments, an intravascular electrode has been described as an example of a device. Nevertheless, a device to be placed in a subject is not limited to the intravascular electrode, and the medical image processing apparatuses 100a to 100d according to the first to the fourth embodiments can be applied to the determination of placement positions of various devices.

Various types of data handled in this specification are typically digital data.

According to at least one embodiment described above, the user can easily recognize a candidate placement position where an intravascular electrode can be placed near an occurrence location of a nerve activity to be measured in the brain.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to:
generate a vessel diameter image indicating a size of a vessel diameter in a brain, from a brain blood vessel image obtained by capturing an image of a blood vessel of the brain;
identify a target position corresponding to a nerve activity, which is a measurement target in the brain, based on an image related to at least either one of a function region of the brain and arrangement of a nerve in the brain, and generate a distance image indicating a distance from the identified target position;
identify a position with both of a vessel diameter and a distance from the target position satisfying a prescribed standard on the blood vessel, based on the vessel diameter image and the distance image, as a candidate placement position of a device; and
display the identified candidate placement position in an intensified manner on an image indicating the brain.

2. The medical image processing apparatus according to claim 1,
wherein the distance image includes a brain function distance image indicating a distance from an activation region of a nerve activity in the brain and a nerve fiber end point distance image indicating a distance from an end point of a nerve fiber to be measured in the brain,
wherein the processing circuitry is further configured to generate the brain function distance image from a brain function image in which the activation region is visualized, and generates the nerve fiber end point distance image from a nerve fiber image in which the nerve fiber in the brain is visualized,
wherein the processing circuitry is further configured to identify the candidate placement position based on the vessel diameter image, the brain function distance image, and the nerve fiber end point distance image, and
wherein the brain blood vessel image, the brain function image, and the nerve fiber image are images obtained by capturing images of a brain of a same subject.

3. The medical image processing apparatus according to claim 1,
wherein the distance image is a brain function distance image indicating a distance from an activation region of a nerve activity in the brain,
wherein the processing circuitry is further configured to generate the brain function distance image from a brain function image in which the activation region is visualized,
wherein the processing circuitry is further configured to identify the candidate placement position based on the vessel diameter image and the brain function distance image, and
wherein the brain blood vessel image and the brain function image are images obtained by capturing images of a brain of a same subject.

4. The medical image processing apparatus according to claim 1,
wherein the distance image is a nerve fiber end point distance image indicating a distance from an end point of a nerve fiber to be measured in the brain,
wherein the processing circuitry is further configured to generate the nerve fiber end point distance image from a nerve fiber image in which the nerve fiber in the brain is visualized,
wherein the processing circuitry is further configured to identify the candidate placement position based on the vessel diameter image and the nerve fiber end point distance image, and
wherein the brain blood vessel image and the nerve fiber image are images obtained by capturing images of a brain of a same subject.

5. The medical image processing apparatus according to claim 1,
wherein the distance image is a brain region distance image indicating a distance from a region from which a nerve activity in the brain is to be measured,
wherein the processing circuitry is further configured to generate the brain region distance image from a brain region atlas indicating anatomical arrangement of a brain region involving the nerve activity, and
wherein the processing circuitry is further configured to identify the candidate placement position based on the vessel diameter image and the brain region distance image.

6. The medical image processing apparatus according to claim 1,
wherein the vessel diameter image is an image indicating a size of a vessel diameter in the brain as a pixel value,
wherein the distance image is an image indicating a distance from the target position as a pixel value, and
wherein the processing circuitry is further configured to identify a position of a pixel with a smallest distance from the target position among pixels with vessel diameters equal to or larger than a prescribed measurement, based on a pixel value of the vessel diameter image and a pixel value of the distance image, as the candidate placement position.

7. The medical image processing apparatus according to claim 1, wherein the processing circuitry is further configured to display a character indicating a vessel diameter at the candidate placement position or a distance from the target position, on an image indicating the brain.

8. The medical image processing apparatus according to claim 1, wherein, the processing circuitry is further configured to, in a case where a plurality of the candidate placement positions satisfying the prescribed standard exist, number the plurality of the candidate placement positions in descending order of vessel diameter, or in ascending order of a distance from the target position, and displays the plurality of the candidate placement positions.

9. The medical image processing apparatus according to claim 1,
wherein, the processing circuitry is further configured to, in a case where a position with both of a vessel diameter and a distance from the target position satisfying the prescribed standard on the blood vessel does not exist, identify a position with either one of a vessel diameter and a distance from the target position satisfying the prescribed standard, as an alternative candidate, and
wherein the processing circuitry is further configured to display the alternative candidate, and a suggestion related to correction of the prescribed standard that is based on a vessel diameter at a position of the alternative candidate and a distance from the target position.

10. The medical image processing apparatus according to claim 1, wherein the processing circuitry is further configured to receive a user operation of correcting the prescribed standard in a case where a position with both of a vessel diameter and a distance from the target position satisfying the prescribed standard on the blood vessel does not exist.
